SEQUENCE LISTING (16 sequences)

SEQ ID NO: 1 - DNA, 43 bp, Artificial sequence
  Primer
ataagaatgcggccgcttttttttttttttttttttttttttt

SEQ ID NO: 2 - DNA, 34 bp, Artificial sequence
  Primer
aaaaaaaaaaaaaaaaaaaaaaaaaaaaaattga

SEQ ID NO: 3 - DNA, 18 bp, Artificial sequence
  Primer
ccrggcggtcccactctt

SEQ ID NO: 4 - DNA, 17 bp, Artificial sequence
  Primer
cagaggctwgtgttacc

SEQ ID NO: 5 - DNA, 45 bp, Parechovirus Ljungan virus
ttgaaaggggggccctgcagccgatataggctgcagggttcccct

SEQ ID NO: 6 - protein, 32 aa, Parechovirus Ljungan virus
CLIAAYMPKSAHDHMDTYTFSSYTNLPHVLMN

SEQ ID NO: 7 - protein, 32 aa, Parechovirus Ljungan virus
CLIAAYMPKSTHDHMDTYTFGSYTNLPHVLMN

SEQ ID NO: 8 - protein, 32 aa, Parechovirus Ljungan virus
CLIAAYMPKTAHDHMNTYTFGSYTNLPHVLMN

SEQ ID NO: 9 - protein, 32 aa, Parechovirus Ljungan virus
CLIACYLPATALENFNSYTFGTFTNFPHVLMN

SEQ ID NO: 10 - protein, 32 aa, Parechovirus Human parechovirus
SALVVYEPKPVVTYDSKLEFGAFTNLPHVLMN

SEQ ID NO: 11 - protein, 61 aa, Parechovirus Ljungan virus
ISTASADELEAVLDAKTPDAPVRLCYQPEDPLRQLREAAKAYFNIMHNDEMDYSGGKFLNQ

SEQ ID NO: 12 - protein, 61 aa, Parechovirus Ljungan virus
ISTASADELEAVLDAKTPDAPVRLCYQPEDPLRQLREAAKAHFNIMHNDEMDYSGGKFLNQ

SEQ ID NO: 13 - protein, 61 aa, Parechovirus Ljungan virus
VDEANAEELEAVLEARTPDAPLRLKFNPEDPLKQLREAAKAYFNIMHSDEMDFAGGKFLNQ

SEQ ID NO: 14 - protein, 61 aa, Parechovirus Ljungan virus
VDEANAEELEAVLEAGTPDAPLRLKFNPEDPLKQLREAAKAYFNIMHSDEMDFAGGKFLNQ

SEQ ID NO: 15 - protein, 61 aa, Parechovirus Ljungan virus
IDTATEEELNAIMSSAEPDTPLRIGKPPEDPLKQLREAAKTYFKIYHDKDMDYAGGKFLNQ

SEQ ID NO: 16 - protein, 16 aa, Parechovirus Human parechovirus
MANLTNQSPGQQPQNR

(19) United States
(12) Patent Application Publication
(10) Pub. No.: US 2012/0027777 A1
(43) Pub. Date: Feb. 2, 2012

(54) LJUNGAN VIRUS WITH IMPROVED REPLICATION CHARACTERISTICS

(75) Inventors: Michael Lindberg, Kalmer (SE); Conny Tolf, Kalmer (SE)

(73) Assignee: APODEMUS AB, Kalmer (SE)

(21) Appl. No.: 13/145,871

(22) PCT Filed: Jan. 22, 2010
(86) PCT No.: PCT/GB10/00103

§ 371 Date: Jul. 22, 2011

(30) Foreign Application Priority Data
Jan. 22, 2009 (GB) 0901056.2

(51) Int. Cl. A61K 39/42; C07H 21/00; C07K 14/085; A61K 39/125; C07K 16/10; C12N 7/04; C07D 413/12; A61K 31/4245; A61P 9/00; A61P 3/10; A61P 25/00; A61P 43/00; A61P 37/04; A61P 31/14; A61P 15/00; C12N 7/00

(52) U.S. Cl. 424/159.1; 435/235.1; 536/23.72; 530/350; 424/216.1; 530/389.4; 435/236; 536/24.32; 536/24.33; 548/131; 514/364

(57) ABSTRACT

The present invention relates to a Ljungan virus with improved replication characteristic and the use of this Ljungan virus, amongst other thing, in the production of a vaccine.

### BACKGROUND OF THE INVENTION

1. Field Of The Invention

The present invention relates to a Ljungan virus with improved replication characteristic and the use of this Ljungan virus, amongst other thing, in the production of a vaccine.

2. Description Of Related Art

Extensive genetic variation is one hallmark of RNA viruses, and consequently these viruses replicate as populations of heterogeneous genetic variants usually referred to as quasispecies [16, 28]. The genetic variation is caused by misincorporations of nucleotides (nt) during RNA synthesis [27] and genomic rearrangements including deletions, insertions and recombination [4]. However, in a constant biological environment such as that provided by a clonal cell population, the selective pressure leads to an optimized replication of the quasispecies population [11, 53, 54]. Picornaviruses are viruses with an icosahedral virion structure and a 7.5-8 kb single-stranded RNA genome of positive polarity [58]. Studies of foot-and-mouth disease virus (FMDV), an animal pathogen of the Aphthovirus genus of Picornaviridae, have shown that hypervirulent viral variants emerge during serial passages in BHK-21 cells [14, 15, 44, 62] Amino acid substitutions associated with hypervirulence have been located to surface exposed regions of the FMDV capsid [15, 26]. In a study of enterovirus type 70 [46], another member of Picornaviridae, causing pandemics of acute hemorrhagic conjunctivitis [8], it was shown that five different amino acid substitutions in the viral capsid affect host range and cytopathogenicity [35]. Several hypotheses have been proposed to explain how individual amino acid substitutions in the viral capsid may convey increased cytopathogenicity, including altered utilization of viral receptor(s) and/or a more efficient induction of apoptosis. Changes of the cellular morphology associated with apoptosis include cell shrinking, cytoplasmic blebbing, chromosome degradation and nuclear fragmentation. Several picornaviruses including poliovirus [2], coxsackievirus B3 [7], enterovirus 71 [36], Theiler's murine encephalomyelitis virus [5, 29] and hepatitis A virus [6] have been associated with induction of apoptosis in cultured cells.

Ljungan virus (LV) is a picornavirus that was discovered twenty years ago in Swedish bank voles (Myodes glareolus) during the search for an infectious agent causing lethal myocarditis in young athletes [48, 49]. To date, the genomes of three Swedish strains and two American strains representing four different genotypes have been isolated from voles of the Myodes and Microtus genera [31, 33, 69]. Human parechovirus (HPeV) forms together with LV the Parechovirus genus within Picornaviridae [39]. Several reports have suggested LV as an etiological agent of diabetes and myocarditis in rodents [48-51]. Recently, LV antigens were detected by immunohistochemistry in fetal tissue samples in cases of human intrauterine fetal death [52].

Initial attempts to propagate LV in BHK-21, Vero and A549 cells resulted in a very subtle cytopathic effect (CPE), corresponding to the effect caused by HPeV infection in cell culture [1, 33]. By serial passages of the LV 87-012 prototype strain in Green Monkey Kidney (GMK) cells, a lytic 87-012G variant evolved [18, 32]. The polyprotein sequence of this variant contained several amino acid substitutions compared to the parental 87-012 strain, but the significance of these substitutions have so far not been studied [18].

In a recent study, the inventors analyzed a cell culture adapted variant of the LV 145SL strain isolated from a Swedish bank vole in the northern part of Sweden [33, 49]. This variant, denoted 145SLG, evolved during serial passages in GMK cells, initially showing a weak and delayed CPE, but turned with increasing number of passages into a virus inducing complete CPE in cells. Genetic analyses showed that the 145SLG and the parental 145SL strain differed by three amino acid substitutions in the viral capsid. Consequently, it was apparent that the introduction of the three amino acid substitutions into the 145SL strain enhanced its replication efficiency and cytopathogenicity.

### SUMMARY OF THE INVENTION

Accordingly, the present invention is directed to variants of the 145SL strain with enhanced replication efficiency and/or cytopathogenicity.

In a first aspect, the present invention provides a 145SL Ljungan virus comprising one or more amino acid substitutions in the viral capsid proteins VP0 and VP1, wherein the one or more amino acid substitutions are selected from alanine-162 to threonine in the VP0 protein, serine-172 to glycine in the VP0 protein, and tyrosine-289 to histidine in the VP1 protein.

As indicated above and in more detail in the exemplary description below, these mutations in the viral capsid proteins of the LV cause the LV to have increased replication properties. This allows easier and quicker production of the virus to higher levels so that more viral particles can be worked on in a single experiment and in a given period of time. This allows larger quantities of virus to be produced for use, for example, in the production of a vaccine.

Characterisation and sequencing of the 145SL strain has previously taken place (33). The nucleotide sequence of the 145SL genome and the amino acid sequence of the 145SL polyprotein can also be found on Genbank (Genbank accession number AF327922, version AF327922.2).

For the sake of clarity, the amino substitutions defined above have the following meaning. For example, an amino acid substitution from alanine-162 to threonine in the VP0 protein means that the naturally occurring amino acid in the VP0 protein of 145SL at residue 162 is alanine which has been substituted with threonine in the variant strain of the invention.

Preferably, the LV comprises two or more amino acid substitutions in the viral capsid proteins VP0 and VP1, wherein the two or more amino acid substitutions are selected from alanine-162 to threonine in the VP0 protein, serine-172 to glycine in the VP0 protein, and tyrosine-289 to histidine in the VP1 protein. More preferably, the LV comprises three amino acid substitutions in the viral capsid proteins VP0 and VP1, wherein the three amino acid substitutions are alanine-162 to threonine in the VP0 protein, serine-172 to glycine in the VP0 protein, and tyrosine-289 to histidine in the VP1 protein.

The change in the nucleotide sequence of the LV can be any suitable change which results in the amino acid substitution indicated above. For example, this may be the result of a single nucleotide substitution such as a change in the alanine codon GCU to the threonine codon ACU. Alternatively, the amino acid substitution may be the result of a plurality of nucleotide substitutions such as a change in the alanine codon GCU to the threonine codon ACA (two nucleotide substitutions) or such as a change in the serine codon UCU to the glycine codon GGC (three nucleotide substitutions). Preferably, the amino acid substitution results from a single nucleotide substitution.

Preferably, the LV of the present invention is isolated, i.e. it is substantially free of its natural environment.

The present invention also provides a nucleotide sequence corresponding to the genomic nucleotide sequence of the LV described above. Therefore, the nucleotide sequence codes for a functional LV with enhanced replication properties. Preferably, the nucleotide sequence is RNA. Preferably, the nucleotide sequence is isolated. This nucleotide sequence can be used, for example, as an antigenic component of a vaccine.

Further, the present invention provides a LV protein selected from:
1) a 145SL VP0 capsid protein comprising an alanine-162 to threonine substitution and/or a serine-172 to glycine substitution; and
2) a 145SL VP1 capsid protein comprising a tyrosine-289 to histidine substitution.

Preferably, the VP0 capsid protein comprises both of the amino acid substitutions. Preferably, the LV protein of the present invention is isolated.

The protein of the invention can be produced rapidly and in high levels through culture of the variant LV of the invention. The protein can be used as an antigenic component of a vaccine.

In addition, the present invention provides a nucleotide sequence encoding for the amino acid sequence of the protein described above. The nucleotide sequence can be any nucleotide sequence which encodes for the amino acid sequence of the protein described above. As one skilled in the art will be aware, there can be several different codons which encode for the same amino acid. For example, there are six different codons (AGU, AGC, UCU, UCC, UCA and UCG) which all encode for the amino acid serine. Accordingly, there are a number of possible nucleotide sequences which all encode for identical proteins. The nucleotide sequence can be either DNA or RNA. Preferably, the nucleotide sequence is isolated. Preferably, the nucleotide sequence is identical to the 145SL sequence except for the nucleotides in the codon encoding for the substituted amino acid. Preferably, the nucleotides in the codon encoding for the substituted amino acid only differ from the 145SL nucleotides by one nucleotide.

The present invention also provides a virus-like particle comprising a VP0, a VP1 and a VP3 capsid protein from a 145SL Ljungan virus, wherein the VP0 and VP1 capsid proteins comprise one or more amino acid substitutions selected from alanine-162 to threonine in the VP0 protein, serine-172 to glycine in the VP0 protein, and tyrosine-289 to histidine in the VP1 protein.

Preferably, the virus-like particle comprises two or more of the selected amino acid substitutions. More preferably, the virus-like particle comprises all three of the selected amino acid substitutions.

This virus-like particle can be produced relatively quickly through culture of the variant LV of the invention and used as an antigenic component of a vaccine. Preferably, the virus-like particle is isolated.

Virus-like particles are particles which resemble the complete virus from which they are derived but lack viral nucleic acid, meaning that they are not infectious. Virus-like particles and methods for producing such particles are well known to those skilled in the art, for example as described in Jennings G. T. and Bachmann M. F. (2008) Biol Chem. 389(5):521-36.

Further, the present invention provides an antibody directed against the protein of the invention defined above. The antibody is specific for the protein so that the antibody only recognises and binds to the protein of the invention and not to other proteins, such as naturally occurring proteins of the 145SL LV which do not contain any amino acid substitutions.

The term “antibody” is well known in the art. Herein it means an immunoglobulin or any functional fragment thereof. It encompasses any polypeptide that has an antigen-binding site. It includes but is not limited to monoclonal, polyclonal, monospecific, polyspecific, non-specific, humanized, human, single-chain, chimeric, synthetic, recombinant, hybrid, mutated, grafted, and in vitro generated antibodies. The term “antibody” encompasses antibody fragments such as Fab, F (ab′) 2, Fv, scFv, Fd, dAb, and any other antibody fragments that retain antigen-binding function. Typically, such fragments would comprise an antigen-binding domain.

Furthermore, the present invention provides a composition for inducing an immune response comprising a component selected from: the LV described above; the LV described above in an attenuated form; the LV described above in an inactivated form; the protein described above; an antigenic fragment of the protein described above; the nucleotide sequence described above; an antigenic fragment of the nucleotide sequence described above; the virus-like particle described above and a combination of two or more of the preceding components.

Preferably, the composition for inducing an immune response is a vaccine. Preferably, the composition comprises an attenuated or inactivated form of the LV of the invention.

The term “antigenic fragment” means any fragment which can stimulate an immune response to the LV. The antigenic fragment can be any size as long as it provokes an immune response which is directed to the LV. For example, an antigenic fragment of a protein could be, for example, a protein subunit, a protein domain, an oligopeptide, etc. The antigenic fragment should be unique to the LV of the invention, e.g., it contains at least one of the three amino acid substitutions or corresponding nucleotide substitutions associated with the amino acid substitutions. For example, an oligopeptide derived from the VP1 capsid protein of the invention will comprise the tyrosine-289 to histidine substitution.

The LV of the invention can be attenuated or inactivated in any suitable way. For example, the LV can be inactivated through treatment with formalin or β-propiolactone (BPL). Suitable methods for inactivating viruses are well known to those skilled in the art and many are described in Stauffer F, et al. (2006) Advances in the development of inactivated virus vaccines. Recent Patents Anti-Infect Drug Disc. 2006 November; 1(3):291-6.

The advantage of the LV of the invention being able to replicate quickly and to high levels means that a large amount of virus, optionally attenuated or inactivated, can be produced in a relatively short time compared to culturing known LVs which replicate slowly and to a low level. This allows immunogenic compositions and vaccines to be produced more easily.

Further, the advantage of the LV of the invention being similar to the 145SL LV is that immunisation with the vaccine or immunogenic composition of the invention should help to protect a mammal against an infection of the LV of the invention and also against an infection of the 145SL LV.

Preferably the composition or vaccine further comprises an adjuvant. Suitable adjuvants are well known to those skilled in the art. The immunogenic composition or vaccine can induce an immune response in a mammal so that infection of the mammal with LV can be efficiently dealt with and eliminated without the mammal suffering from a disease associated with infection by the LV.

Diseases associated with LVs can be any of myocarditis, cardiomyopathia, Guillain Barre syndrome, diabetes mellitus, multiple sclerosis, chronic fatigue syndrome, myasthenia gravis, amyothrophic lateral sclerosis, dermatomyositis, polymyositis, spontaneous abortion, intrauterine fetal death, lethal central nervous disease and sudden infant death syndrome.

The present invention also provides a diagnostic kit comprising a component selected from: an antibody described above; a nucleotide probe directed against a unique portion of the genome of the LV described above; and specific primers for specific amplification of a portion of the genome of the LV described above.

The nucleotide probe can be any kind of probe which can bind to a unique portion of the genome of the LV to allow detection of the LV RNA. This unique portion will comprise the nucleotide substitutions that result in the one or more amino acid substitutions. Since the nucleotide probe is directed against a unique portion of the genome of the LV, it will be specific for the LV so that it will not bind to the genome of other LVs and other viruses. The nucleotide probe can be an oligonucleotide with a complementary sequence to the sequence contained in the portion of the viral genome against which the probe is directed. For example, the oligonucleotide could be DNA, RNA, a phosphorodiamidate morpholino oligo (PMO), a 2′O-Me oligonucleotide or a locked nucleic acid (LNA). Preferably, the nucleotide probe is at least a 10 mer, more preferably, at least a 20 mer and, most preferably, at least a 30 mer. Suitable probes are well known to those skilled in the art and could easily be produced based on the sequence of the LV.

Preferably, the antibody or the probe are tagged or labelled with a molecular marker to allow the antibody or the probe to be easily detected. Suitable tags or labels are well known to those skilled in the art. For example, the antibody or probe may be labelled with a radioactive isotope such as ³²P.

The primers can be any suitable primers for amplifying a portion of the genome of the LV described above using, for example, PCR. Based on the sequence of the LV, a person skilled in the art would be able to create specific primers to allow the detection of the LV of the invention. The primers are specific for the LV of the invention, i.e. at least one primer binds to a unique portion of the genome of the LV. This allows detection of only the LV of the invention so that other viruses are not detected, thereby avoiding a false positive result. Suitable primers could easily be produced by one skilled in the art based on the sequence of the LV. Preferably, the primers are between 10 and 30 nucleotides in length.

This allows the diagnostic kit to be used to identify the LV of the invention, for example, LV infection in a mammal. The kit can comprise one, two or all three of the components discussed above.

In another aspect, the present invention provides a pharmaceutical composition comprising a component selected from: the LV described above; the LV described above in an attenuated form; the LV described above in an inactivated form; the protein described above; an antigenic fragment of the protein described above; the nucleotide sequence described above; an antigenic fragment of the nucleotide sequence described above; the antibody described above; the virus-like particle described above and the immunogenic composition described above for use in therapy.

The present invention also provides a pharmaceutical composition comprising a component selected from: the LV described above; the LV described above in an attenuated form; the LV described above in an inactivated form; the protein described above; an antigenic fragment of the protein described above; the nucleotide sequence described above; an antigenic fragment of the nucleotide sequence described above; the antibody described above; the virus-like particle described above and the immunogenic composition described above for use in the prophylactic or therapeutic treatment of a disease caused by the LV of the invention.

The disease caused by the LV can be any of myocarditis, cardiomyopathia, Guillain Bane syndrome, diabetes mellitus, multiple sclerosis, chronic fatigue syndrome, myasthenia gravis, amyothrophic lateral sclerosis, dermatomyositis, polymyositis, spontaneous abortion, intrauterine fetal death, lethal central nervous disease and sudden infant death syndrome.

Pharmaceutical compositions according to the invention comprise the component described above with any pharmaceutically acceptable carrier, adjuvant or vehicle. Suitable pharmaceutically acceptable carriers, adjuvants and vehicles are well known to those skilled in the art. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered orally, parenterally, by inhalation spray, topically, rectally, nasally, buccally, vaginally or via an implanted reservoir. Preferably, they are administered orally or by injection. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the antigen is combined with emulsifying and suspending agents. If desired, certain sweetening and/or flavouring and/or colouring agents may be added.

The present invention also provides a method of prophylactic or therapeutic treatment of a disease caused by the LV described above in a mammal, the method comprising administering to the mammal a prophylactically or therapeutically effective amount of a pharmaceutical composition comprising a component selected from: the LV described above; the LV described above in an attenuated form; the LV described above in an inactivated form; the protein described above; an antigenic fragment of the protein described above; the nucleotide sequence described above; an antigenic fragment of the nucleotide sequence described above; the antibody described above; the virus-like particle described above and the immunogenic composition described above.

The mammal can be any mammal which can be infected with LV and in which LV can cause disease. Preferably, the mammal is human.

The present invention also provides a method of prophylactic and/or therapeutic treatment of a mammal for a disease that is caused by infection with the LV of the invention, comprising administration to said mammal of an antivirally effective amount of an antiviral compound effective against the LV to eliminate or inhibit proliferation of said virus in said mammal and at the same time prevent and/or treat said disease in said mammal.

Preferably the mammal is selected from the group consisting of humans, horses, cattle, pigs, cats, dogs and rodents such as rats and mice.

The disease caused by LV infection may be caused by the infection of a tissue or cell type. It is known that LV is capable of growth in most cell types of the body and can therefore infect all organs of the body.

The present invention also provides an antiviral compound effective against a LV of the invention for use in the treatment of a disease in a mammal that is caused by infection of the LV of the invention.

Preferably, the antiviral compound is Pleconaril or a derivative thereof which is effective against the LV of the invention. Suitable antiviral compounds, such as Pleconaril and derivatives thereof, are set out in International Patent Application WO2004/073710.

### BRIEF DESCRIPTION OF THE DRAWINGS

The invention will now be described in detail, by way of example only, with reference to the accompanying figures in which:

FIG. 1 shows schematic representations of genomes of the LV 145SL strain and the 145SLG variant as well as recombinant variants derived from these viruses as they are inserted in the pCR-Script Direct SK+ vector. Indicated designations of viral cDNA clones and virus derived from these clones (given within parenthesis) are used throughout the article. The 5′ and 3′ UTRs and the P1, P2 and P3 genome regions of the open reading frame (represented by open boxes) encoding the LV polyprotein are indicated. The genomic positions of the restriction enzymes utilized for construction of the viral cDNA clones are referring to positions in the 145SLG genome. Differences in the amino acid sequences between the 145SLG and the 145SL strain are indicated next to VP0-, VP1-, 2B- and 3A-encoding genes of the 145SLG genome. The first letter is referring to the residue of the 145SL proteins and given numbers is referring to residues in the individual proteins.

FIG. 2 shows the characterization of viral replication for LV 145SL and 145SLG. (A) Viral replication of 145SL and 145SLG measured by real time PCR. The replication was determined as the fold change of viral plus sense RNA between the time of infection and at 72 h post infection. The amount of viral RNA at indicated time points were determined by the cycle threshold (Ct) value from the real time PCR amplification. The fold change of viral RNA is expressed as mean values±standard deviation. (B) One-step growth analysis for 145SLG. Virus titers at different time points post infection were determined by plaque assay titration on GMK cells. The cells were infected with virus at a MOI of 1 and the number of plaques was determined 8 days post infection. Viral titers are expressed as number of plaques of triplicate 3.5 cm² wells±standard deviation.

FIG. 3 shows evaluation of LV replication in GMK cells determined by the cytopathic effect (A), detection of viral antigens (B) and plaque morphology (C). (A) Monolayers of GMK cells were subjected to virus derived from indicated viral cDNA clones and the cytopathic effect was monitored at indicated time points. (B) The synthesis of viral antigens was analyzed using an antibody against the LV VP1 capsid protein. Cells were fixed at indicated time points post infection and viral antigens were visualized using an Alexa 488-conjugated antibody (green stain) and the nuclei in cells were stained by DAPI (blue). (C) Plaque morphology of indicated viruses was determined 10 days post infection. Viral plaques were visualized by neutral red staining of viable GMK cells.

FIG. 4 shows viral titers determined for 145SL, 145SLG and their recombinant derivatives. Titers are expressed as the mean log TCID₅₀/ml of triplicate samples (i.e. each virus was derived from indicated viral cDNA clones by three parallel transfections followed by one blind passage)±standard deviation. Viral titers were determined by 10-fold titrations on GMK cells in six parallel wells of the 96-well culture plate. (*) No evident CPE was observed in cells infected with the 145SL strain.

FIG. 5 shows the apoptotic response in GMK cells infected with 145SL, 145SLG and the recombinant variants of these viruses, as determined by caspase-3 activation (A) and DNA degradation (B) assays. (A) Cells subjected to depicted LV variants were fixed at indicated time points and assayed for apoptotic response using an antibody reactive to cleaved/activated caspase-3. Antigen-antibody complexes in cells were visualized by an Alexa 488-conjugated antibody (green stain) and the nuclei of GMK cells were stained using DAPI (blue stain). (B) Electrophoresis assay of DNA degradation in GMK cells infected with indicated viruses. Cells were harvested at 48 h post infection and the nucleic acids were isolated as described in the method section. The positive control sample was generated by treatment of uninfected GMK cells with staurosporine. M, molecular weight markers and numbers indicate the molecular masses (bp). Mock-infected cells were included as negative control.

FIG. 6 shows predicted locations in LV VP0 and VP1 capsid proteins of the 145SLG substitutions that influence cytopathogenicity in GMK cells. Predicted β-strands surrounding the EF-loop of the LV and HPeV1 VP0 protein are underlined. The dotted line indicates a cleavage site that was predicted initially during molecular characterization of the LV genome [33]; however, subsequent analyses of the LV capsid proteins have indicated that cleavage at this site does not occur [32, 67].

### DETAILED DESCRIPTION OF THE INVENTION

In a recent study, the inventors analyzed a cell culture adapted variant of the LV 145SL strain isolated from a Swedish bank vole in the northern part of Sweden [33, 49]. This variant, denoted 145SLG, evolved during serial passages in GMK cells, initially showing a weak and delayed CPE, but turned with increasing number of passages into a virus inducing complete CPE in cells. Genetic analyses showed that the 145SLG and the parental 145SL strain differed by three amino acid substitutions in the viral capsid. The association between these substitutions in the VP0 and VP1 capsid protein and changes of replication efficiency and cytopathogenicity was analyzed by a reverse genetic approach including viral cDNA copies of the parental LV 145SL and the 145SLG variant. In addition, significance of individual substitutions was analyzed using recombinant constructs of the viral full-length clones, encoding 145SLG capsid substitutions in a 145SL genome. Furthermore, the association between increased viral replication efficiency mediated by capsid substitutions and indication of apoptosis was studied in cultured cells.

### Methods

### Cells and Viruses

A local variant of GMK cells were propagated in Dulbecco's modified Eagle's medium (DMEM) supplemented with 10% newborn calf serum (NCS), 1% L-glutamine and 1% penicillin-streptomycin (PEST). The LV 145SL strain was propagated in brains of suckling mice as previously reported [33, 49]. Initially, replication of 145SL in cell culture was characterized by a delayed, barely detectable CPE. However, by serial passages in GMK cells, this virus was adapted to an efficient cytolytic replication.

### Sequence Analysis of the LV 145SLG Genome

Prior to sequence analysis, the 145SLG variant was propagated by two passages in GMK cells. Infections were allowed to proceed in 37° C. until complete CPE was observed. Following a second passage, viruses were released from cells by three rounds of freeze-thawing, as previously described [18]. Viral RNA was extracted from 140 μl of infected cell lysate using the QIAamp viral RNA mini kit (Qiagen) according to the manufacturer's instructions. cDNA was generated by subjecting extracted RNA to reverse transcription (RT) using Superscript III (Invitrogen) and the primer NotdT₂₇ (5′-ATAAGAATGCGGCCGCT₂₇-3′). RT reactions were incubated at 50° C. for 1 h and the enzyme was inactivated at 70° C. Primers derived from the LV 145SL sequence and the PicoMaxx high fidelity PCR system (Stratagene) were used to amplify the LV cDNA. Resulting specific and overlapping amplicons were isolated by agarose gel electrophoresis and sequenced by a primer walking strategy on both strands as previously described [33]. The 5′-end sequence of the 5′UTR was obtained using a modified variant of the 5′ rapid amplification of cDNA ends (5′RACE) method [19], as described previously for the LV 87-012G virus [18]; although, instead of using NotdA₂₇ as the sense primer to amplify the polyT-tailed cDNA, a dA₃₀TTGA-primer was used.

Construction of Infectious Viral cDNA Clones

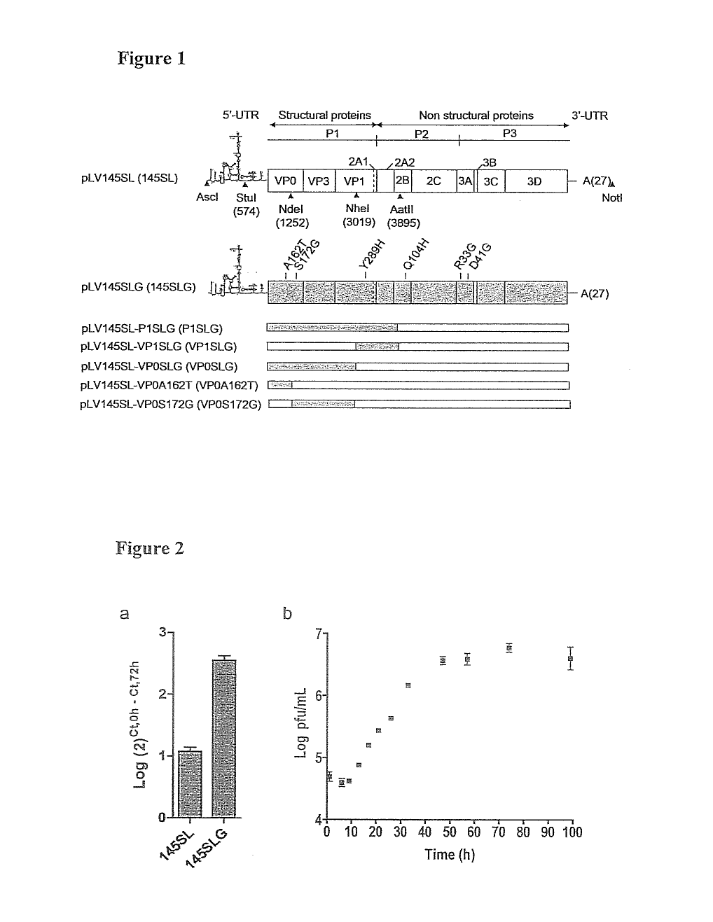

Two full-length infectious viral cDNA clones were constructed (FIG. 1). The genome sequences of the 145SL strain [33] and the 145SLG variant (reported here) were cloned into the pCR-Script Direct SK+vector (Stratagene) as described previously [18, 38, 57]. In the pLV145SL and pLV145SLG cDNA clones, the complete viral genome sequences are flanked by the restriction endonuclease sites AscI upstream the 5′-end of the LV genomes and the Nod site downstream a poly-A tail of 27 adenosines (FIG. 1). The nucleotide sequences of constructed cDNA clones were verified by sequencing as described above. Replicating viruses generated by transfection of pLV145SL and pLV145SLG will be denoted as 145SL and 145SLG, respectively. In order to analyze the effect of identified amino acid substitutions in the P1 region of the cell culture adapted 145SLG variant (including amino substitutions A162T and S172G in VP0 and Y289H in the VP1 structural protein, see Table 1) on viral replication efficiency and cytopathogenicity, five different recombinant viral clones were constructed (FIG. 1). The restriction enzyme cleavage site StuI and AatII in the pLV145SL and pLV145SLG plasmids were used to produce a clone containing all three 145SLG P1 mutations named pLV145SL-P1SLG (P1SLG), where a simplified name of virus derived from the clone is given within parenthesis (FIG. 1). The StuI and NheI cleavage sites were used in order to construct the pLV145SL-VP0SLG clone (VP0SLG), containing the A162T and S172G substitutions of LV 145SLG VP0, while the NheI and AatII sites were used to generate the pLV-VP1SLG clone (VP1SLG), containing the VP1 Y289H substitution. In order to analyze the cellular effect of the individual 145SLG VP0 substitutions, the StuI and NdeI sites of pLV145SL and pLV145SLG clones were used to construct the pLV145SL-VP0A162T clone (VP0A162T), while the NdeI and NheI sites were used for the pLV145SL-VP0S172G clone (VP0S172G). The nucleotide sequences of recombinant LV constructs were verified by sequencing.

Generation and Propagation of Virus from Viral cDNA Clones

Viral plasmids were purified from bacteria using Midiprep kit (Promega). Purified plasmid were precipitated, dissolved in nuclease-free water and quantified using a spectrophotometer (NanoDrop ND-100, Saveen Werner, Sweden). The different LV cDNA constructs were transfected into GMK cells using 2.5 μg of the viral plasmids and Lipofectamine 2000 (Invitrogen). Transfected cells were maintained in 37° C. for five days and generated virus were harvested from cells by three rounds of freeze-thawing [18]. The clone derived viruses were further propagated by one blind passage in GMK cells as published previously [45]. Briefly, confluent monolayers of GMK cells in T25 flasks, maintained in DMEM supplemented with 1% NCS, 1% L-glutamine and 1% penicillin-streptomycin, were inoculated with 0.5 mL of lysates from cells transfected with the different LV 145SL/SLG cDNA clone constructs. After this passage, the nucleotide sequences of the replicating viral variants were determined to verify the presence of cloned mutations and to ensure that no additional mutations had been introduced during the procedure. Finally, the viruses were harvested by freeze-thawing and stored in -20° C. until they were characterized in subsequent analyses. In order to study morphological effects in cells hosting replicating viruses of LV 145SL/SLG cDNA clones and their derivatives, GMK cells were infected as described above. Morphological changes in GMK cells subjected to the different LV variants were visualized by microscopic images at 24, 72 and 168 h post infection (p.i.) and compared with the morphology of mock-infected cells (mock).

### Analysis of 145SL and 145SLG Replication by Real Time PCR

The replication of LV plus strand RNA within cells infected with the 145SL or 145SLG was determined using real-time PCR as previously described [18]. Briefly, confluent GMK cells in 24-well plates were infected with 0.1 mL of virus infected cell lysates. Virus was allowed to bind to cells for 1 h at 37° C., and was then replaced with 0.5 mL DMEM supplemented with 1% NCS, 1% L-glutamine and 1% penicillin-streptomycin Immediately after addition of the cell culturing media, samples denoted 0 h for 145SL and 145SLG infected cells were collected by three rounds of freeze-thawing, while samples denoted 72 h were incubated at 37° C. for 3 days before they were collected. Viral RNA was isolated from collected samples and reverse transcribed using TaqMan transcriptase kit (Applied Biosystems) according to the manufacturer's instruction and random hexamers. Viral cDNA were generated from three parallel infections with virus derived from the pLV145SLG clone, while two cDNA samples were analyzed for infection with virus derived from the pLV145SL clone. Real-time PCR was performed using Power SYBR Green master mix (Applied Biosystems) and the LV-250 (5′-CCRGGCGGTCCCACTCTT-3′) and LV-251 (5′-CAGAGGCTWGTGTTACC-3′) primers. These primers generate a 187 by amplicon within the 5′ untranslated region (5′UTR) of the 145SLG genome. Each cDNA was analyzed in triplicates by real time PCR reactions using the Applied Biosystems 7500 Real-Time PCR System (Applied Biosystems) with the standard cycle setup [18]. The starting copy number of cDNA (i.e. viral RNA) in analyzed samples was determined by cycle threshold (Ct) values and the difference between time of infection (0 h) compared to 72 h p.i. was expressed as the fold change [(2)^{Ct,0h Ct,72h}], approximating 100% efficiency for the real time PCR amplification according to the 2^{-ΔΔCt} method [41].

### Plaque Assay and One-Step Growth Analysis for the 145SLG Strain

Plaque morphology of the 145SL and 145SLG variants as well as the 145SLG growth kinetics were determined by plaque assay titration as previously described [18], with minor modifications. Briefly, virus generated from cDNA clones were titrated by 10-fold dilutions onto confluent monolayers of GMK cells in six-well plates. After adsorption for 1 h at 37° C., the inoculum was aspired and 3 ml of an overlay containing 1.5% SeaPlaque GTG agarose (Lonza) in DMEM supplemented with 1% NCS, 1% L-glutamine and 1% PEST was added. The overlay was allowed to solidify for 30 min at room temperature before they were incubated for 8-10 days at 37° C. Cells were fixed with 10% formaldehyde dissolved in phosphate buffered saline (PBS) for 1 h at room temperature. The formaldehyde was aspired and the overlay was removed before viable cells were stained using a neutral red stain for 1 h at room temperature.

One-step growth curves for the clone derived 145SLG variant were determined by infection of monolayers of GMK cells (approximately 10⁶ cells/3.5 cm²plate) with virus at a multiplicity of infection (MOI) of 1. After incubation on cells for 1 h at 37° C., the virus solution was removed and the cells were washed three times with DMEM and then replaced with 2 ml of fresh DMEM supplemented with 1% L-glutamine and 1% PEST. Cells incubated at 37° C. were collected at indicated times points post infection by three rounds of freeze-thawing. Viral titers were determined by plaque assay as described previously in this section. Each time point of the growth curve represents the mean value of triplicate samples and the variations of mean values are given as standard deviations.

### Detection of LV by Immunofluorescence Microscopy

For immunofluorescence assays, confluent monolayers of GMK cells cultured on Lab-TEK II chamber glass slides (Nalge Nunc International) were infected with clone derived virus. A 25 μl volume of 145SLG infected cell lysate and 250 μl of virus infected lysates from remaining viral clones were used to initiate infections. These inoculations correspond to a MOI of approximately 2 for the 145SLG and P1SLG variant, down to approximately 0.01 for the VP0A162T virus. Hence, this was not intended as a comparative replication assay for the different viral variants, but merely a way to establish translation of viral antigens within infected cells. After adsorption on cells for 2 h at 37° C., the cells were washed with DMEM before adding 400 μl of fresh DMEM supplemented with 5% NCS, 1% L-glutamine and 1% PEST. Cells were incubated at 37° C. and were at indicated time points washed with PBS and fixed by treatment with cold 4% formaldehyde-PBS solution for 30 min. Background fluorescence were minimized by blocking fixed cells with 5% NCS dissolved in PBS containing 0.1% Tween 20, and slides were incubated with a polyclonal antibody (70 nM) against the LV VP1 capsid protein to detect LV infected cells [67], or for the analyses of cellular apoptosis, with a polyclonal antibody (1:500) against cleaved/activated caspase-3 (Cell Signaling Technology). The primary antibodies, dissolved in PBS containing 0.1% bovine serum albumin and 0.1% Tween 20, were incubated on cells for 1 h in room temperature. The slides were washed three times with PBS and incubated with a secondary antibody labeled with Alexa Fluor® 488 (Molecular Probes). Finally, slides were mounted with Vectashield, containing DAPI (Immunokemi, Sweden), and images were captured using an epifluorescence microscope (Nikon).

### Virus Infectivity Assay

Virus production and cytopathogenicity of 145SL and 145SLG viruses as well as the P1 region recombinants were determined by TCID₅₀ titration. Subconfluent GMK cells in 96-well plates were infected with 100 μl of 10-fold serial titrations of each virus followed by incubation at 37° C. for 7 days. Viruses generated by three parallel transfections for each cDNA clone were assayed, and each virus dilution was analyzed on cells in six parallel wells. Viral titers were determined based on visual signs of CPE in the microscope. The TCID₅₀/ml-values was calculated using the Spearman Kärber method [24].

### Analyses of Cellular DNA Degradation

Degradation of chromosomal DNA is one of the hallmarks of apoptotic cells [17]. DNA degradation in GMK cells infected with 145SL and 145SLG as well as recombinant derivatives of these viruses was assayed as previously described by [68] with some minor modifications. Briefly, subconfluent GMK cells in T25-flasks were infected with 0.5 mL of virus lysates generated from viral cDNA clones by transfection and subsequent blind passage as previously described. LV infected and mock-infected cells were harvested 48 h post infection using Versene buffer (0.5 mM EDTA and 10 ∞M glucose in PBS, pH 7.4) and collected by low-speed centrifugation. Lysis of cells was induced using 0.2% Triton X-100, 0.5 mM EDTA in 10 mM Tris-HCl, pH 7.4. Fragmented chromatin was released from cell nuclei by vortexing. The nucleic components were separated (20,000×g for 10 min at 4° C.) and the nucleic acids in the supernatant fraction were precipitated. After precipitation, the nucleic acids were washed in 70% ethanol and dissolved in 50 μl TE buffer (1 mM EDTA, 10 mM Tris-HCl, pH 7.4). The RNA fraction of isolated nucleic acids was degraded using RNase A (10 μg/ml for 30 min at 37° C.). Finally, 1 μg isolated nucleic acids of each sample were separated by electrophoresis in a 1.5% agarose gel. Apoptosis in control cells was induced by a 16 h treatment of GMK cells with 0.5 μM staurosporine (Sigma), a broad spectrum protein kinase inhibitor [10]. The nucleic acids from these control cells were otherwise treated in the same way as described for cells infected with LV.

### Location of 145SLG Substitutions in Predicted Secondary Structures of the VP0 and VP1 Capsid Proteins

Secondary structures of the LV and HPeV1 capsid proteins were predicted by using the Jpred 3 and PSIPRED 2.6 methods [13, 34]. These programs use neural networks or modified example-based learning algorithms, and structures are based on a set of aligned homologous sequences, either provided by the user or generated by the PSI-BLAST server (http://blast.ncbi.nlm.nih.gov/Blast.cgi?PAGE=Proteins). Both types of alignments were used and evaluated for the structure predictions. In addition, the predicted secondary structure of the VP0 capsid protein was adjusted to homologous sequences (indicated by the Jpred 3 server) of the Theiler's murine encephalomyelitis virus strain Bean (GenBank accession number P08544, Protein Data Bank accession number 1tmf) and the Cricket paralysis virus (P13418, 1b35), and was also compared with previously predicted secondary structure of HPeV1 VP0 [20, 64].

### Results

Sequence Analysis of the LV 145SLG Genome and Construction of Infectious Viral cDNA Clones

Initial attempts to passage the parental LV 145SL strain, isolated from a bank vole (Myodes glareolus), were characterized by a mild CPE barely distinguishable from the morphology of uninfected cells [33, 49]. The complete 145SL genome sequence, except for the most 5′-proximal part of the 5′UTR, was previously reported [33]. By repeated passages in cell culture, the 145SL virus was adapted to cytolytic replication in GMK cells, and the complete genomic sequence of this 145SLG variant was determined from overlapping PCR amplicons (GenBank accession number FJ384560). In order to facilitate construction of infectious viral cDNA clones of the 145SL strain and of the cell culture adapted 145SLG variant, the complete 5′UTR of the 145SLG strain was determined using a previously reported 5′-RACE method [18]. By this method, 45 additional nucleotides were obtained in the 5′ terminal part of the 5′UTR sequence (Table 1). However, the putative four initial nucleotides of the 5′ end could not be determined despite several attempts. Thus, the proposed TTAG tetranucleotide sequence in the reported 5′ end of the 145SLG genome is derived from the LV prototype variant 87-012G for which the complete genome sequence has been determined [18]. The secondary structure of the 5′ termini of the 5′UTR of the 145SLG strain, including the additional 45 nucleotides, was predicted (data not shown). The obtained structure was almost identical to the folding predicted for the corresponding regions of the cell culture adapted LV 87-012G variant of the LV prototype [18]. The additional 5′UTR sequence facilitated construction of full-length cDNA clones of the 145SLG variant and the parental 145SL strain,

The complete sequence of the 145SLG genome (7657 nucleotides excluding the poly A tail) includes a single open reading frame (ORF), which encodes a polyprotein of 2256 amino acids. The genome also includes a 5′ (755 nucleotides) and 3′ (107 nucleotides) untranslated region (UTR) and the genome ends with a 3′ -terminal poly A tail. No sequence variation between the 5′ and 3′UTR of 145SL strain and 145SLG variant was observed. Analysis of the polyprotein derived from the 145SLG genome sequence revealed six amino acid substitutions compared to the parental 145SL strain (Table 1). Three of these substitutions were located in the structural VP0 and VP1 proteins, while the three remaining substitutions were located in the non-structural 2B and 3A proteins (FIG. 1). In addition, two synonymous mutations were observed in the 145SLG genome sequence (Table 1). The limited number of amino acid changes between the 145SLG and the parental 145SL strain allowed investigations of how individual amino acid substitutions may affect the cytolytic phenotype of 145SLG by employing reverse genetics and recombinant full-length cDNA copies of the original 145SL strain and the lytic 145SLG variant. Initial analyses using 145SL cDNA recombinants encoding the P1, P2 or P3 polyprotein regions (FIG. 1) of the 145SLG variant showed that 145SLG substitutions in the viral capsid (the P1 region) were associated with cytolytic replication in GMK cells. Therefore, the focus of this study was to evaluate the significance of the individual Al 62T and S172G substitutions of the VP0 protein and the Y289H substitution of the VP1 protein (Table 1). To enable reverse genetic analyses of the capsid substitutions, recombinants of the pLV145SL and pLV145SLG were constructed. In recombinant viral cDNA clones, one, two or all three mutations of the 145SLG P1 region were introduced in the parental 145SL genome (FIG. 1).

TABLE 1
Determined mutations and corresponding amino
acid substitutions in the LV 145SLG genome
compared to the previously reported
LV 145SL strain [33].

| | | Amino acid |
|---|---|---|
| Genomic region | Mutation^{b} | substitution^{c} |
| 5′ untranslated | | —^{d} |
| region^{a} | | |
| VP0 | C-960→T | —^{e} |
| | G-1243→A | Ala-162→Thr |
| | A-1273→G | Ser-172→Gly |
| VP1 | C-2307→T | — |
| | T-3133→C | Tyr-289→His |
| 2B | G-3936→C | Gln-104→His |
| 3A | A-5143→G | Arg-33→Gly |
| | A-5168→G | Asp-41→Gly |

^{a}45 additional nucleotides (TTGAAAGGGGGGCCCTGCAGCCGATATAGGCTGCAGGGTTCCCCT) have been included in the 5′ untranslated region of the 145SLG genome compared to the parental 145SL strain, but the initial TTGA nucleotides are derived from the completely determined 5′UTR sequence of the LV 87-012G strain [18].
^{b}The first letter is referring to the nucleotide of the parental 145SL strain. Given numbers are the nucleotide positions in the 145SLG genome.
^{c}The first amino acid is the one of the 145SL protein and the residues are numbered independently for each protein.
^{d}Not applicable
^{e}Synonymous mutation.

### Quantification of Viral RNA Synthesis for 145SL and 145SLG Variant

Replication of the 145SL strain induces a very weak CPE in GMK cells [33]. Therefore, in order to verify active replication of virus derived from the pLV145SL cDNA clone (the original 145SL type), the accumulation of viral positive sense RNA during the course of infection was measured by real time PCR as previously published [18]. The accretion of viral RNA during replication of 145SL and 145SLG were analyzed. By comparing the cycle threshold (Ct)-values at the beginning of infection and at 72 h p.i., an increase of viral RNA was determined (FIG. 2a). These results demonstrate that both 145SL and 145SLG replicate in GMK cells. However, during the first 72 h of infection, the increase of viral plus sense RNA is approximately 30 times higher for the cell culture adapted 145SLG variant compared to the parental 145SL strain (FIG. 2a). Taken together, these data shows that the full-length clones are biologically active and that the 145SLG viruses replicate, based on the measured increase of viral RNA during infection, more efficiently in GMK cells than the original 145SL strain.

### One-Step Growth Analysis of the 145SLG Strain

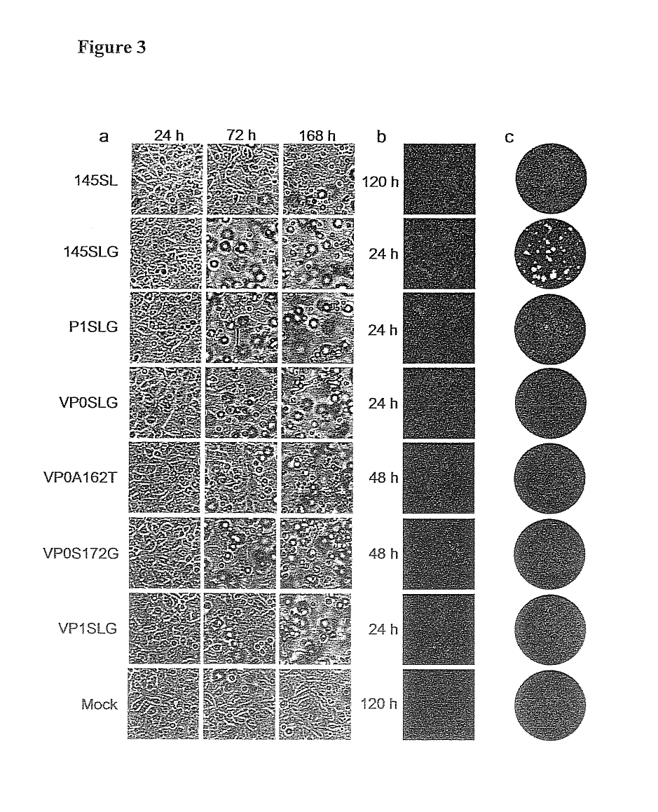

Previously, the replication capacity of the cell culture adapted LV 87-012G variant was characterized by one-step growth analyses [18]. The clearly visible plaques of 145SLG (FIG. 3c) made a corresponding analysis possible for this virus (FIG. 2b). After an initial eclipse phase of 9 h, the 145SLG titer increases exponentially for the next 30 h. The titer peak at 47 h p.i. of approximately 10⁷ plaque forming units (pfu)/ml, coincides in time with initial signs of CPE (data not shown). These results differ from those obtained for the LV 87-012G strain, for which viral titers ceased to increase approximately 18 h p.i., although the first signs of CPE in the GMK cells were not observed until 2-3 days later [18]. The replication dynamics recorded by the one-step growth curve of 145SLG enabled us to decide at what time point it would be most suitable to analyze ongoing active viral replication of 145SLG variants in cultured cells.

Evaluation of Infections with Virus Derived from Constructed cDNA Clones

Cellular effects caused by replicating virus derived from the viral cDNA clones of the 145SL and 145SLG variants as well as recombinants of these viruses were analyzed in GMK cells (FIG. 3). Infected cells were monitored for 7 days (168 h). As expected, no or very weak signs of infection were observed in cells subjected to 145SL (FIG. 3a). In addition, the replication of 145SL could not be detected at 5 days p.i. using an antibody directed against the LV VP1 capsid protein or by formation of plaques at 10 days p.i. (FIGS. 3b and 3c). This is consistent with previous observations of the LV 145SL strain and the LV 87-012 prototype [18, 33, 49]. In contrast, 145SLG caused, already at 48 h p.i., a clearly distinguishable CPE, which was progressing until the entire monolayer of GMK cells were disrupted at 72 h p.i. (FIG. 3a and data not shown). Furthermore, the replication of the 145SLG was also verified by detection of viral antigens at 24 h p.i. and by formation of clearly visible plaques at 10 days p.i. (FIGS. 3b and 3c). A similar result was observed for the P1SLG variant (i.e. a virus encoding non-structural proteins of the 145SL and a P1 region derived from the 145SLG variant). However, the CPE induced by P1SLG was progressing at a slower pace compared to the 145SLG strain; thus, still at 72 h p.i. approximately 30-50% of the cells appeared viable (FIG. 3a). Nonetheless, four days later (168 h p.i.), all GMK cells were disrupted by replicating P1SLG virus. Moreover, in cells infected with this virus, viral antigens were detected at 24 h p.i., but plaques were significantly smaller than those formed by the 145SLG variant. For viruses encoding both VP0 amino acid replacements (VP0SLG) or a single substitution (VP1SLG, VP0A162T and VP0S172T), signs of CPE in GMK cells were observed at 72 h p.i., although in an inferior number of cells than observed for 145SLG and P1SLG (FIG. 3a). Still, eventually, as depicted by microscopic images captured 7 days p.i., these viruses induce complete CPE in GMK cells (FIG. 3a). Also, viral antigens were detected at 24-48 h p.i. in cells infected with recombinant virus expressing one or two of the 145SLG P1 substitutions (FIG. 3b), but the plaques formed by these viruses were very small, barely visible and very difficult to reproduce in images (FIG. 3c). These results suggest that each one of the individual substitutions in the VP0 and VP1 capsid proteins of the cell culture adapted variant 145SLG contributes to an increased cytopathogenicity in GMK cells.

### Quantification of Cytolytic Replication of 145SL and 145SLG Variants

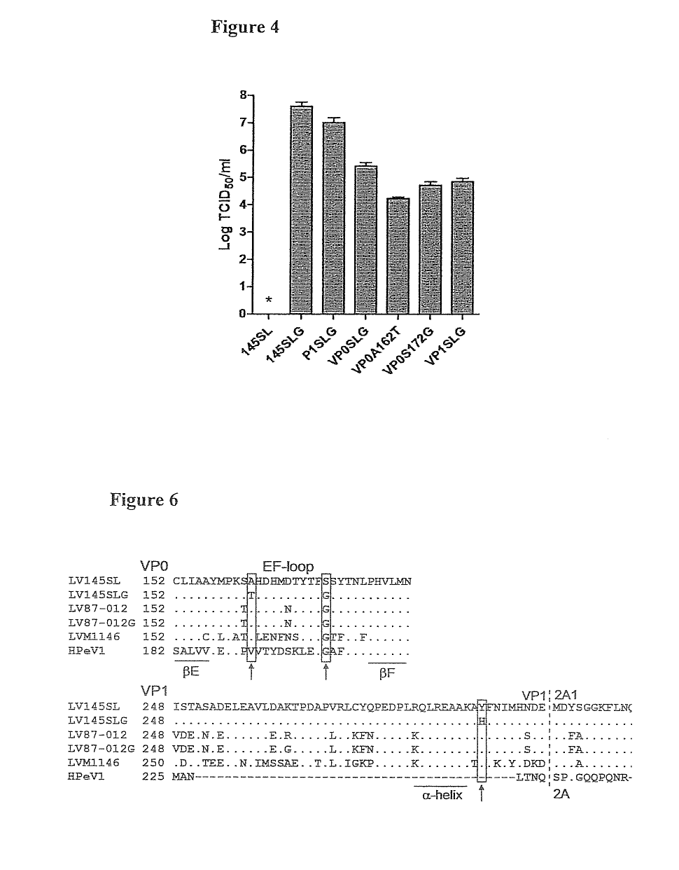

The significance of the individual A162T, S172S and Y289H substitutions of the 145SLG VP0 and VP1 proteins for replication efficiency and cytopathogenicity in cell culture was quantified by virus titration on GMK cells (FIG. 4). Due to the small plaque phenotype displayed by several of the recombinant variants of 145SL and 145SLG it was not possible to use pfu to determine virus titers. Hence, viral titers were determined using the TCID₅₀ method. As shown in previous evaluation of the virus infection (FIG. 3), no or very CPE was observed in cells infected with parental 145SL virus. Because of this absence of evident CPE no virus titer could be determined for 145SL (FIG. 4). However, the titer values for virus encoding a single 145SLG P1 substitution (VP0A162T, VP0S172G and VP1SLG) show that each one of these substitutions result in a change from the non-lytic infection of 145SL into a cytolytic replication that is possible to quantify in GMK cells (FIG. 4). In addition, the two A162T and S172G substitution expressed by VP0SLG appears to function cooperatively since the titer value determined for this virus is approximately 10 times higher than the virus with the single VP0 A162T mutation and five times higher than a virus expressing the S172G substitution. In addition, a virus containing all three substitutions of the 145SLG capsid (P1SLG) replicates to a titer of approximately 10⁷ TCID₅₀/ml, which is approximately 100 times higher than titers for recombinant viruses encoding single capsid protein substitutions (FIG. 4).

Cytolytic LV Infection in GMK Cells is Associated with Caspase-3 Activation and DNA Degradation

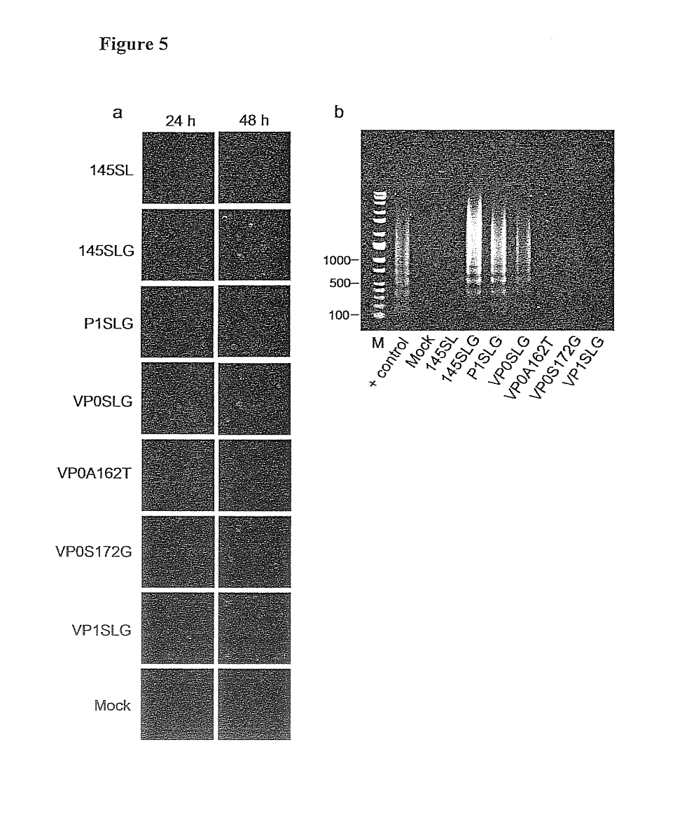

Infection by a variety of different viruses induces host cell responses associated with induction of apoptosis [55, 61]. Therefore, studies on whether LV infections are associated with an apoptotic cellular response were initiated. Caspase-3 activation and DNA fragmentation are important markers for cells entering apoptosis [17]. While no signs of apoptosis were observed during non-cytolytic replication of 145SL, infection of GMK cells with recombinant viruses encoding one or more of the 145SLG P1 substitutions resulted in a cell response where inactive pro-caspase was processed into activated caspase-3, a clear sign of apoptosis (FIG. 5a). Another hallmark of apoptosis is cleavage of chromosomal DNA into 200 by internucleosomal fragments [17]. Administration of staurosporine, an inducer of apoptosis and thus also DNA fragmentation, to GMK cells resulted in expected processing of the nuclear DNA (FIG. 5b), but no DNA fragmentation was observed from mock-infected cells. Infection of GMK cells with the parental 145SL strain did not result in DNA fragmentation. However, nuclear DNA extracted from 145SLG infected cells at 48 h p.i. showed a clear pattern of fragmentation (FIG. 5b). Moreover, the recombinant viruses expressing either the amino acid replacements of VP0 or all three amino acid substitutions of the 145SLG capsid also induced DNA fragmentation at 48 h p.i. in GMK cells. Cells infected with viruses containing one of the 145SLG P1 substitutions also exhibited DNA fragmentation, but less pronounced at the time point studied. These results are in accordance with the observed data regarding how rapid and efficient different recombinant 145SLG variants proceed to full CPE, where viruses containing a single P1 substitution eventually cause a complete CPE although the first signs of cytolysis is not observed before 72 h post infection. What particular cell death a certain picornavirus induces is depending on a variety of viral and cellular factors. However, the efficiency by which a virus replicates may exert a decisive effect [59]. Comparison of results from the caspase-3 activation and DNA degradation assays (FIGS. 5a and 5b) with the viral titer values for the 145SL and 145SLG viral variants (FIG. 4), suggest that there is a correlation between viral replication efficiency and the induction of caspase-3 activation and DNA fragmentation in GMK cells.

### Putative Location of 145SLG Substitutions in the VP0 and VP1 Capsid Proteins

The secondary structure of the LV capsid proteins were predicted using the Jpred and PSI-PRED methods, which were then adjusted to determined or predicted structures of homologous viral sequences [20, 42, 64, 66]. In predicted structures (FIG. 6), the A162T and S172G substitutions of the 145SLG VP0 capsid protein were mapped to a surface exposed structure, corresponding to the EF-loop of the VP2 protein of enteroviruses [25, 60]. The Y289H replacement is located the carboxy-terminal part of the VP1 capsid protein of 145SLG, which is a 43 amino acid extension compared to the most closely related HPeV, and adjacent to the 2A1 amino acid motif (FIG. 6). The size of this extension is conserved in all LV strains sequenced so far, although the sequence variation within this region between different LV strains is extensive (FIG. 6). The carboxy-terminal part of picornavirus VP1 is frequently exposed at the virion surface in determined capsid structures, but the unique insertion in this region of LV VP1 makes structure predictions difficult. However, the extensive sequence variation in this region of VP1 among different LV strains indicates that it is partly exposed on the surface of the virion and may be subjected to host immunity.

### Discussion

LV was initially isolated from Swedish bank voles [48, 49]. First passages of LV in cell culture resulted in a weak and delayed CPE [33], resembling the CPE of HPeV, but clearly distinct from the lytic effect normally induced by enterovirus [70]. However, with increasing number of passages in GMK cells a lytic variant of the LV 87-012 strain evolved, inducing a clearly visible CPE at 3-4 days p.i. [18, 39]. Later on, the LV 145SL strain, initially growing at very low titers without evident signs of CPE, was adapted to a productive replication in GMK cells inducing complete CPE. This cell culture adapted variant was denoted LV 145SLG. The sequence of this variant was determined, including 41 additional nucleotides in the 5′-end of the 5′UTR. Computer-based secondary structure prediction of the most 5′-terminal end of the 5′UTR region suggested that these additional nucleotides are forming a stem-loop structure, almost identical to predicted SL-A1 of LV 87-012G [18]. Although the particular function of this conserved structure is presently not known, its significance was illustrated by a viral DNA copy of the LV 87-012G virus in which the SL-A1 genome region was truncated. This deletion proved non-viable for the LV prototype [18]. Sequence analysis showed that the LV 145SL and 145SLG polyprotein sequences differ by six amino acid residues. Three of these substitutions are located in the P1 region within the VP0 and the VP1 proteins of the 145SLG capsid. By reverse genetic analyses using recombinant viral 145SL cDNA clones, encoding the capsid substitutions of the 145SLG variant, it was shown that these capsid proteins replacements are major determinants of a viral phenotype causing cytolysis during replication. Comparative structural analysis indicates that the A162T and S172G substitutions of the VP0 protein map to a putative capsid structure referred to as the EF-loop. In the β-barrel structure of picornavirus capsid proteins, the EF- and BC-loops are exposed on the surface of the virion and the sequence variation of these exposed structures renders them immunogenic [18, 47, 60]. The Y289H substitution was mapped to carboxy-terminal part of the LV VP1 capsid protein. No homologous picornavirus sequences can presently be used to model this region (data not shown). However, according to secondary structure prediction by the Jpred and PSIPRED software, the VP1 substitution is located adjacent to an α-helix including eight residues. The extensive sequence variation in this VP1 region between different LV strains suggests that it is at least partly exposed to host immunity in the surface of the virion.

The significance of identified 145SLG capsid substitutions for increased replication efficiency and cytopathogenicity was analyzed using infectious DNA copies of the 145SL and 145SLG genomes. Recombinant clones were constructed where one, two or all three of the 145SLG P1 mutations were introduced in the 145SL genome. One-step growth of the LV 145SLG virus, generated by transfection of the pLV145SLG clone, showed a virus production in GMK cells corresponding to the end-point titers of the LV 87-012G variant [33, 39]. However, the LV 87-012G variant reach its end-point titer (˜10⁷ pfu/ml) within the first 24 h p.i., which is one day faster compared to LV 145SLG. Sixteen amino acid changes were observed within the genome of the LV 87-012G variant compared to the parental LV 87-012 strain [18]. Eight of these replacements are located in the viral capsid, but no substitutions were identified in the EF-loop of the VP0 protein corresponding to the Al 62T and S172G changes of the LV 145SLG virus. At present, it is not known if the substitutions in the capsid of the LV 87-012G variant contribute to its increased replication efficiency and cytopathogenicity in GMK cells. Replication of the LV 145SLG variant in GMK cells was distinguished at 24 h p.i. by detection of viral antigens, but also by the first signs of CPE, a cytopathogenicity that disrupted all cells within the next 48 h. In contrast, all detection methods but PCR failed detecting replication of the LV 145SL, indicating as previously shown [33] that GMK cells are nearly non-permissive for replication of the parental virus. However, as suggested by analyses of virus derived from the recombinant 145SL clones, the expression of one of the VP0 or VP1 capsid substitution of the 145SLG variant is sufficient to transform 145SL into a virus with elevated replication efficiency and cytopathogenicity. Moreover, the results presented in this work also suggest that accumulation of the 145SLG capsid substitutions gradually increase viral replication efficacy. Hence, the viral titer determined for P1SLG virus, a virus encoding all three 145SLG P1 substitutions, is almost the same as for the 145SLG variant. There are several possible explanations for the observed differences of replication efficiency and cytopathogenicity in cell culture between the parental 145SL strain and the 145SLG variant. The viral receptor usage of the 145SL strain in GMK cells might not permit efficient cell attachment and entry resulting in restricted replication and without evident signs of CPE. The differences in cytopathogenicity might also be due to intracellular interactions leading to apoptosis. Different picornavirus proteins have been associated with the induction of apoptosis, including the VP1 of foot-and-mouth disease virus [56], the VP2 of coxsackievirus B3 [23], the VP3 of avian encephalomyelitis virus [40] as well as the enterovirus 2A and 3C proteases [9, 21, 36, 37]. In GMK cells infected with the 145SLG variant or the 145SLG recombinants, DNA degradation as well as caspase-3 activation were observed, both hallmarks of the apoptotic pathway. These results suggest that induction of CPE might be associated with an apoptotic response within infected GMK cells. For poliovirus and Theiler's murine encephalomyelitis virus, optimized cell culturing systems are available in which these viruses are highly cytolytic causing rapid cell destruction. The interplay between a picornavirus infection, cell necrosis and apoptosis is however so far less clear. If the viral growth are markedly depressed due to mutations or by replication in less permissive cells, the cell may respond by induction of apoptosis [29, 30, 59, 63].

Replication of LV 87-012G and 145SLG variants, adapted to lytic replication in GMK cells produces a CPE that is similar to the cytolysis induced by HPeV, but distinct from the usually observed CPE induced during productive infection by enterovirus [1, 3, 70]. The reason for these differences in cytopathogenicity and growth kinetics between LV and HPeV on one hand and enteroviruses on the other hand is not known. One factor that possibly contributes to these differences are that several picornaviruses including enteroviruses inhibit translation of cell mRNAs by proteolytic cleavage of cellular translation initiation factors [22, 43, 65]. In cells infected with LV and HPeV, no specific inhibition of translation of cellular mRNA has been observed [12, 18, 64]. Hence, in LV infected cells, there is a competition between endogenous translation of cellular mRNAs and internal ribosome entry site (IRES)-mediated translation of viral polyproteins. This competition will probably interfere with viral growth and will probably late in infection, as suggested above for non-premissive infections by poliovirus and Theiler's murine encephalomyelitis virus, lead to induction of apoptosis. Whether differences between LV and viruses within the Enterovirus genus depends on specific genetic traits dictating the kinetics of viral replication and cellular response, or if these difference is simply a matter of optimization of the virus-host cell culturing system is a question to be answered in future studies of how LV replicate and are able to adapt to different environments.

### REFERENCES

1. Abed Y et al., 2006, Emerg. Infect. Dis., 12: 969-975
2. Agol V I et al.,1998, Virology, 252: 343-353
3. Agol V I et al., 2000, J. Virol., 74: 5534-5541
4. Agol V I, 2002, Mol. Biol.,(Mosk), 36: 286-295
5. Anderson R et al., 2000, Brain Res., 868: 259-267
6. Brack K et al., 1998, J. Virol., 72: 3370-3376
7. Carthy C M et al., 1998, J. Virol., 72: 7669-7675
8. Chang C H et al., 2003, Graefes Arch. Clin. Exp. Ophthalmol., 241: 554-560
9. Chau D H et al., 2007, Apoptosis, 12: 513-524
10. Chow J M et al., 2006, Apoptosis, 11: 1999-2011
11. Clarke D K et al., 1993, J. Virol., 67: 222-228
12. Coller B A et al., 1990, J. Virol., 64: 2692-2701
13. Cuff J A et al., 2000, Proteins, 40: 502-511
14. de la Tone J C et al., 1985, Virology, 145: 24-35
15. Diez J et al., 1990, J. Virol., 64: 5519-5528
16. Domingo E et al., 1997, Annu. Rev. Microbiol., 51: 151-178
17. Edinger A L et al., 2004, Curr. Opin. Cell. Biol., 16: 663-669
18. Ekström J O et al., 2007, Virus Res., 130: 129-139
19. Frohman M A et al., 1988, Proc. Natl. Acad. Sci. USA, 85: 8998-9002
20. Ghazi F et al., 1998, J. Gen. Virol., 79 (Pt 11): 2641-2650
21. Goldstaub D et al., 2000, Mol. Cell. Biol., 20: 1271-1277
22. Gradi A et al., Proc. Natl. Acad. Sci. USA, 95: 11089-11094
23. Henke A et al., 2000, J. Virol., 74: 4284-4290
24. Hierholzer J C et al., 1996, In Mahy B W J, Kangro H O (ed) Virology Methods Manual Academic Press, San Diego, pp 25-47
25. Hogle J M et al., 1985, Science, 229: 1358-1365
26. Holguin A et al., 1997, J. Gen. Virol., 78 (Pt 3): 601-609
27. Holland J et al., 1982, Science, 215: 1577-1585
28. Holland J J et al., 1992, Curr. Top. Microbiol. Immunol., 176: 1-20
29. Jelachich M L et al., 1996, J. Virol., 70: 6856-6861
30. Jelachich M L et al., 1999, J. Gen. Virol., 80 (Pt 7): 1701-1705
31. Johansson E S et al., 2003, J. Gen. Virol., 84: 837-844
32. Johansson E S et al., 2004, Biochem. Biophys. Res. Commun , 317: 1023-1029
33. Johansson S et al., 2002, J. Virol., 76: 8920-8930
34. Jones D T, 1999, J. Mol. Biol., 292: 195-202
35. Kim M S et al., 2007, J. Virol., 81: 8648-8655
36. Kuo R L et al., 2002, J. Gen. Virol., 83: 1367-1376
37. Li M L et al., 2002, Virology, 293: 386-395
38. Lindberg A M et al., 1997, J. Virol. Methods, 65: 191-199
39. Lindberg A M et al., 2002, Virus Res., 85: 61-70
40. Liu J et al., 2002, Virology, 300: 39-49
41. Livak K J et al., 2001, Methods, 25: 402-408
42. Luo M et al., 1992, Proc. Natl. Acad. Sci. USA, 89: 2409-2413
43. Maltese E et al., 2000, J. Gen. Virol., 81: 1373-1381
44. Martinez M A et al., 1997, Proc. Natl. Acad. Sci. USA, 94: 6798-6802
45. Minor P D et al., 1985,1 Gen. Virol., 66 (Pt 5): 1159-1165
46. Miyamura K et al., 1986, Arch. Virol., 89: 1-14
47. Muckelbauer J K et al., 1995, Structure, 3: 653-667
48. Niklasson B et al., 1998, Emerg. Infect. Dis., 4: 187-193
49. Niklasson B et al., 1999, Virology, 255: 86-93
50. Niklasson B et al., 2003, Int. J. Exp. Diabesity Res., 4: 35-44
51. Niklasson B et al., 2003, Ann. N Y Acad. Sci., 1005: 170-175
52. Niklasson B et al., 2007, Birth Defects Res. A. Clin. Mol. Teratol, 79: 488-493
53. Novella I S et al., 1995, J. Virol., 69: 6805-6809
54. Novella I S et al., 1995, Proc. Natl. Acad. Sci. USA, 92: 5841-5844
55. O'Brien V, 1998, J. Gen. Virol., 79 (Pt 8): 1833-45
56. Peng J M et al., 2004, J. Biol. Chem., 279: 52168-74
57. Polacek C et al., 1999, Virus Res., 59: 229-238
58. Racaniello V R, 2001, In: Straus SE (ed) Fields virology. Lippincott Williams & Wilkins, Philadelphia, pp 658-722
59. Romanova L I et al., 2005, Virology, 331: 292-306
60. Rossmann M G et al., 1985, Nature, 317: 145-153
61. Roulston A et al., 1999, Annu. Rev. Microbiol., 53: 577-628
62. Sevilla N et al., 1996, J. Virol., 70: 6617-6624
63. Son K N et al., 2008, J. Virol., 82: 4502-4210
64. Stanway G et al., 1994, J. Virol., 68: 8232-8238
65. Svitkin Y V et al., 1999, J. Virol., 73: 3467-3472
66. Tate J et al., 1999, Nat. Struct. Biol., 6: 765-774
67. Tolf C et al., 2008, J. Virol. Methods, 150: 34-40
68. Tolskaya E A et al., 1995, J. Virol., 69: 1181-1189
69. Whitney E et al., 1970, J. Wildl. Dis., 6: 48-55
70. Wigand R et al., 1961, Arch. Gesamte. Virusforsch, 11: 224-247

1. A 145SL Ljungan virus (LV) comprising one or more amino acid substitutions in the viral capsid proteins VP0 and VP1, wherein the one or more amino acid substitutions are selected from alanine-162 to threonine in the VP0 protein, serine-172 to glycine in the VP0 protein, and tyrosine-289 to histidine in the VP1 protein.

2. The virus of claim 1 comprising two or more of the selected amino acid substitutions in the viral capsid proteins VP0 and VP1.

3. The virus of claim 1 comprising all three of the selected amino acid substitutions in the viral capsid proteins VP0 and VP1.

4. A nucleotide sequence corresponding to the genomic nucleotide sequence of the LV of claim 1.

5. A LV protein selected from: a 145SL VP0 capsid protein comprising an alanine-162 to threonine substitution, a serine-172 to glycine substitution or both; and a 145SL VP1 capsid protein comprising a tyrosine-289 to histidine substitution.

6. A nucleotide sequence encoding for the protein of claim 5.

7. A virus-like particle comprising a VP0, a VP1 and a VP3 capsid protein from a 145SL LV, wherein the VP0 and VP1 capsid proteins comprise one or more amino acid substitutions selected from alanine-162 to threonine in the VP0 protein, serine-172 to glycine in the VP0 protein, and tyrosine-289 to histidine in the VP1 protein.

8. An antibody directed against the protein of claim 5, wherein the antibody is specific for the protein.

9. A composition for inducing an immune response comprising a component selected from: a 145SL Ljungan virus (LV) comprising one or more amino acid substitutions in the viral capsid proteins VP0 and VP1, wherein the one or more amino acid substitutions are selected from alanine-162 to threonine in the VP0 protein, serine-172 to glycine in the VP0 protein, and tyrosine-289 to histidine in the VP1 protein; said LV in an attenuated form; said LV in an inactivated form; a nucleotide sequence corresponding to the genomic sequences of said LV; an antigenic fragment of the nucleotide sequence; the LV protein selected from: a 145SL VP0 capsid protein comprising an alanine-162 to threonine substitution, a serine-172 to glycine substitution or both; and a 145SL VP1 capsid protein comprising a tyrosine-289 to histidine substitution; an antigenic fragment of said LV protein; a virus-like particle comprising a VP0, a VP1 and a VP3 capsid protein from a 145SL LV, wherein the VP0 and VP1 capsid proteins comprise one or more amino acid substitutions selected from alanine-162 to threonine in the VP0 protein, serine-172 to glycine in the VP0 protein, and tyrosine-289 to histidine in the VP1 protein; and a combination of two or more of the preceding components.

10. The composition of claim 9, wherein the composition is a vaccine.

11. The composition of claim 9 comprising an attenuated or inactivated said LV.

12. The composition of claim 9, further comprising an adjuvant.

13. A diagnostic kit comprising a component selected from: an antibody specific for an LV in protein selected from: a 145SL VP0 capsid protein comprising an alanine-162 to threonine substitution, a serine-172 to glycine substitution or both; and a 145SL VP1 capsid protein comprising a tyrosine-289 to histidine substitution; a nucleotide probe directed against a unique portion of the genome of the LV; and specific primers for amplification of a portion of the genome of the LV.

14. A pharmaceutical composition comprising a component selected from: a 145SL Ljungan virus (LV) comprising one or more amino acid substitutions in the viral capsid proteins VP0 and VP1, wherein the one or more amino acid substitutions are selected from alanine-162 to threonine in the VP0 protein, serine-172 to glycine in the VP0 protein, and tyrosine-289 to histidine in the VP1 protein; said LV in an attenuated form; said LV in an inactivated form; a nucleotide sequence corresponding to the genomic sequences of said LV; an antigenic fragment of the nucleotide sequence; the LV protein selected from: a 145SL VP0 capsid protein comprising an alanine-162 to threonine substitution, a serine-172 to glycine substitution or both; and a 145SL VP1 capsid protein comprising a tyrosine-289 to histidine substitution; an antigenic fragment of said LV protein; a virus-like particle comprising a VP0, a VP1 and a VP3 capsid protein from a 145SL LV, wherein the VP0 and VP1 capsid proteins comprise one or more amino acid substitutions selected from alanine-162 to threonine in the VP0 protein, serine-172 to glycine in the VP0 protein, and tyrosine-289 to histidine in the VP1 protein; and a combination of two or more of the preceding components; an antibody specific for said protein; and a 145SL Ljungan virus (LV) comprising one or more amino acid substitutions in the viral capsid proteins VP0 and VP1, wherein the one or more amino acid substitutions are selected from alanine-162 to threonine in the VP0 protein, serine-172 to glycine in the VP0 protein, and tyrosine-289 to histidine in the VP1 protein; said LV in an attenuated form; said LV in an inactivated form; a nucleotide sequence corresponding to the genomic sequences of said LV; an antigenic fragment of the nucleotide sequence; the LV protein selected from: a 145SL VP0 capsid protein comprising an alanine-162 to threonine substitution, a serine-172 to glycine substitution or both; and a 145SL VP1 capsid protein comprising a tyrosine-289 to histidine substitution; an antigenic fragment of said LV protein;
a virus-like particle comprising a VP0, a VP1 and a VP3 capsid protein from a 145SL LV, wherein the VP0 and VP1 capsid proteins comprise one or more amino acid substitutions selected from alanine-162 to threonine in the VP0 protein, serine-172 to glycine in the VP0 protein, and tyrosine-289 to histidine in the VP1 protein; and a combination of two or more of the preceding components for use in therapy.

15. A pharmaceutical composition comprising a component selected from: a composition for inducing an immune response comprising a component selected from: a 145SL Ljungan virus (LV) comprising one or more amino acid substitutions in the viral capsid proteins VP0 and VP1, wherein the one or more amino acid substitutions are selected from alanine-162 to threonine in the VP0 protein, serine-172 to glycine in the VP0 protein, and tyrosine-289 to histidine in the VP1 protein; said LV in an attenuated form; said LV in an inactivated form; a nucleotide sequence corresponding to the genomic sequences of said LV; an antigenic fragment of the nucleotide sequence; a 145SL VP0 capsid protein comprising an alanine-162 to threonine substitution, a serine-172 to glycine substitution or both; and a 145SL VP1 capsid protein comprising a tyrosine-289 to histidine substitution; an antigenic fragment of said LV protein; a virus-like particle comprising a VP0, a VP1 and a VP3 capsid protein from a 145SL LV, wherein the VP0 and VP1 capsid proteins comprise one or more amino acid substitutions selected from alanine-162 to threonine in the VP0 protein, serine-172 to glycine in the VP0 protein, and tyrosine-289 to histidine in the VP1 protein; and a combination of two or more of the preceding components; an antibody specific for said protein; a 145SL Ljungan virus (LV) comprising one or more amino acid substitutions in the viral capsid proteins VP0 and VP1, wherein the one or more amino acid substitutions are selected from alanine-162 to threonine in the VP0 protein, serine-172 to glycine in the VP0 protein, and tyrosine-289 to histidine in the VP1 protein; said LV in an attenuated form; said LV in an inactivated form; a nucleotide sequence corresponding to the genomic sequences of said LV; an antigenic fragment of the nucleotide sequence; the LV protein selected from: a 145SL VP0 capsid protein comprising an alanine-162 to threonine substitution, a serine-172 to glycine substitution or both; and a 145SL VP1 capsid protein comprising a tyrosine-289 to histidine substitution; an antigenic fragment of said LV protein; a virus-like particle comprising a VP0, a VP1 and a VP3 capsid protein from a 145SL LV, wherein the VP0 and VP1 capsid proteins comprise one or more amino acid substitutions selected from alanine-162 to threonine in the VP0 protein, serine-172 to glycine in the VP0 protein, and tyrosine-289 to histidine in the VP1 protein; and a combination of two or more of the preceding components for use in the prophylactic or therapeutic treatment of a disease caused by said LV.

16. The pharmaceutical composition of claim 15, wherein the disease is selected from myocarditis, cardiomyopathia, Guillain Bane syndrome, diabetes mellitus, multiple sclerosis, chronic fatigue syndrome, myasthenia gravis, amyothrophic lateral sclerosis, dermatomyositis, polymyositis, spontaneous abortion, intrauterine fetal death, lethal central nervous disease and sudden infant death syndrome.

17. A method of prophylactic or therapeutic treatment of a disease caused by a 145SL Ljungan virus (LV) comprising one or more amino acid substitutions in the viral capsid proteins VP0 and VP1, wherein the one or more amino acid substitutions are selected from alanine-162 to threonine in the VP0 protein, serine-172 to glycine in the VP0 protein, and tyrosine-289 to histidine in the VP1 protein in a mammal, the method comprising administering to the mammal a prophylactically or therapeutically effective amount of a pharmaceutical composition comprising a component selected from: a composition for inducing an immune response comprising a component selected from: a 145SL Ljungan virus (LV) comprising one or more amino acid substitutions in the viral capsid proteins VP0 and VP1, wherein the one or more amino acid substitutions are selected from alanine-162 to threonine in the VP0 protein, serine-172 to glycine in the VP0 protein, and tyrosine-289 to histidine in the VP1 protein; said LV in an attenuated form; said LV in an inactivated form; a nucleotide sequence corresponding to the genomic sequences of said LV; an antigenic fragment of the nucleotide sequence; the LV protein selected from: a 145SL VP0 capsid protein comprising an alanine-162 to threonine substitution, a serine-172 to glycine substitution or both; and a 145SL VP1 capsid protein comprising a tyrosine-289 to histidine substitution; an antigenic fragment of said LV protein; a virus-like particle comprising a VP0, a VP1 and a VP3 capsid protein from a 145SL LV, wherein the VP0 and VP1 capsid proteins comprise one or more amino acid substitutions selected from alanine-162 to threonine in the VP0 protein, serine-172 to glycine in the VP0 protein, and tyrosine-289 to histidine in the VP1 protein; and a combination of two or more of the preceding components; an antibody specific for said protein; and a composition for inducing an immune response comprising a component selected from: a 145SL Ljungan virus (LV) comprising one or more amino acid substitutions in the viral capsid proteins VP0 and VP1, wherein the one or more amino acid substitutions are selected from alanine-162 to threonine in the VP0 protein, serine-172 to glycine in the VP0 protein, and tyrosine-289 to histidine in the VP1 protein; said LV in an attenuated form; said LV in an inactivated form; a nucleotide sequence corresponding to the genomic sequences of said LV; an antigenic fragment of the nucleotide sequence; the LV protein selected from: a 145SL VP0 capsid protein comprising an alanine-162 to threonine substitution, a serine-172 to glycine substitution or both; and a 145SL VP1 capsid protein comprising a tyrosine-289 to histidine substitution; an antigenic fragment of said LV protein; a virus-like particle comprising a VP0, a VP1 and a VP3 capsid protein from a 145SL LV, wherein the VP0 and VP1 capsid proteins comprise one or more amino acid substitutions selected from alanine-162 to threonine in the VP0 protein, serine-172 to glycine in the VP0 protein, and tyrosine-289 to histidine in the VP1 protein; and a combination of two or more of the preceding components for use in therapy.

18. A method of prophylactic and/or therapeutic treatment of a mammal for a disease that is caused by infection with the LV of claim 1, comprising administration to said mammal of an antivirally effective amount of an antiviral compound effective against the LV to eliminate or inhibit proliferation of said virus in said mammal and at the same time prevent and/or treat said disease in said mammal.

19. An antiviral compound effective against the LV of claim 1 for use in the treatment of a disease in a mammal that is caused by infection of the LV.

20. The method of claim 18, wherein the antiviral compound is Pleconaril or a derivative thereof.

21. The compound of claim 19, wherein the antiviral compound is Pleconaril or a derivative thereof.
